(19) United States
(12) Patent Application Publication
(10) Pub. No.: US 2022/0304564 A1
(43) Pub. Date: Sep. 29, 2022

(54) A ROBOT FOR ENDOSCOPY

(71) Applicant: University of Dundee, Dundee (GB)

(72) Inventor: Luigi Manfredi, Dundee (GB)

(21) Appl. No.: 17/619,920

(22) PCT Filed: Jun. 19, 2020
(86) PCT No.: PCT/EP2020/067230

§ 371 Date: Dec. 16, 2021

(30) Foreign Application Priority Data
Jun. 22, 2019 (GB) 1908949.9

(51) Int. Cl. A61B 1/31; A61B 1/00; A61B 1/04; A61B 34/32; A61B 1/018

(52) U.S. Cl. CPC A61B 1/31; A61B 1/00156; A61B 1/00148; A61B 1/041; A61B 1/00103; A61B 34/32; A61B 1/018; A61B 2034/301

(57) ABSTRACT

This invention relates to a robot to improve the inspection of a pipe or lumen. More particularly, this invention relates to a soft-pneumatic robot for endoscopy that is suitable for performing colonoscopy procedures. The robot of this invention comprises leading and tailing balloons configured for selective inflation to anchor a portion of the robot within the pipe or lumen and a soft-pneumatic actuator that is located between the leading balloon and the trailing balloon. The soft-pneumatic actuator is operable to extend or contract the robot in a longitudinal direction, in addition to positioning the leading balloon in a 360 degree circumference around the longitudinal direction by pneumatically controlling a fluid within a plurality of chambers within the soft-pneumatic actuator.

### FIELD OF THE INVENTION

Embodiments of the present invention relate to a robot for endoscopy or, more generally, for inspection of a pipe or lumen. Particularly, but not exclusively, embodiments of the invention relate to a robot suitable for colonoscopy.

### BACKGROUND OF THE INVENTION

Colorectal cancer (CRC) is the 3rd most common cause of cancer death worldwide. Regular screening of the asymptomatic population can drastically reduce the mortality rate. CRC screening includes several procedures although the gold standard remains optical colonoscopy (OC) because of its high sensitivity and low false negative rate. However, OC is unpleasant and causes pain and discomfort for the subject. New technologies exemplified by capsule endoscopy (CE) constitute alternative painless solutions and despite their limitations (for example, passive locomotion and absence of on-board instrumentation), are being increasingly used for CRC screening.

Typically, colonoscopy is performed with a flexible 1.6 meter long colonoscope by a trained colonoscopist. A colonoscope is a semi-flexible tube with two cable-driven degrees of freedom (DOF) in the tip (pitch and yaw angles), which are controlled by an external manual handle. Two additional DOF are provided by rotating the colonoscope via its handle and by pushing the instrument through the anus. This pushing force is required to introduce and navigate the colonoscope inside of the lumen of the colon to reach the caecum (top expanded section). During this procedure, the long and passive tube is pushed against the colonic wall, inducing pain and discomfort for the patient. For this reason, colonoscopy for screening and diagnosis of symptomatic diseases may often require sedation and analgesia. Aside from discomfort, rare instances of colonic perforation requiring emergency surgery to prevent peritonitis remain a concern.

Cleaning and chemical sterilization of colonoscopes are necessary as the device is expensive, non-disposable and requires periodic servicing and maintenance to ensure a continued optimal function.

In addition, conventional optical colonoscopy is a difficult procedure requiring a long period of training (2-3 years on average) for acquisition of the required level of proficiency for safe expert execution and interpretation.

U.S. Pat. No. 5,337,732 discloses an endoscopic robot having a number of segments surrounded by balloons and joined together through solid articulated joint links. Actuators within the segments can move the segments together and apart to allow the robot to move in an inchworm fashion along a lumen. In one embodiment, four inflatable sacs are employed within a segment, each having an internal pressure sensor, such that control of the relative pressure distribution in the sacs can cause the robot to bend. However, this design requires internal electronics, complex construction and solid joints which all contribute to making the robot costly, time-consuming to produce, difficult to maintain and limited in flexibility.

U.S. Pat. No. 7,935,047 discloses a double-balloon endoscope system comprising a shape detection apparatus for detecting positions of respective points in an insertion section of the endoscope. However, this design includes internal electro-magnetic parts which, again, make the device, costly, time-consuming to produce and difficult to maintain.

It is therefore an aim of the present invention to provide a robot for endoscopy that ameliorates one or more of the above problems or at least provides a useful alternative.

### SUMMARY OF THE INVENTION

In general terms, the present disclosure proposes a robot for endoscopy, pipe or lumen inspection or colonoscopy and which is configured as a soft-pneumatic inch-worm double-balloon device (SPID).

Soft robotics is a relatively new research area where soft materials (e.g. elastomers) are used to build robots rather than using traditional rigid materials. As an advantage, the robot is safer than a rigid one because of a low Young's modulus, which makes the material softer and reduces the impact and any damage if in contact with human tissues or organs.

An advantage in using such a small robot to perform colonoscopy is that once inserted through the anus, the device can travel by its intrinsic locomotion capability to the caecum virtually abolishing pain and discomfort, as it avoids pressure on the colonic wall and mesenteric tending by loop formation.

The robot may include a camera in its distal end for visual inspection and, optionally, instruments for treatment. A soft tether may be employed for high quality video transmission to an external user console for powering and control of the robot. The tether also provides a safety mechanism for withdrawing the robot in case of malfunction.

The friction provided by the tether against the colonic wall is a drag force in the locomotion. To overcome this force the robot needs to provide enough locomotive traction, although this can be challenging considering the small size and light weight of such a robot and the slippery colonic mucosa.

The more conventional approach to designing robots for colonoscopy is essentially by construction of components made of rigid miniaturized mechanical and electronic parts, which may require expensive high precision machining. Thus, such a rigid robotic colonoscope must be re-usable so that the device will be a financially viable proposition. Even if fully developed, it is most unlikely that such a robot would significantly reduce the costs of screening colonoscopy for colorectal cancer.

The use of soft materials has the advantage of reducing the forces applied to the colonic wall and consequently diminishing pain and discomfort to the patient during the procedure. Because of low mechanical stiffness, a soft robot can perform dexterous movements and follow the 3D-shaped contours of the colonic lumen without the need of a complex active closed-loop control. An additional advantage of using soft materials is the low production costs. The body of the robot can be produced by “injection moulding” at a very low cost, enabling the robot to be configured as a disposable device and thus avoiding the issues of cleaning, disinfection, and maintenance. This will drastically reduce health care costs compared to the traditional optical colonoscopy whilst also increasing the overall acceptability of the procedure by patients.

Design of a soft robot for colonoscopy is challenging because of the limited space within the lumen of the colon, which typically has an internal diameter ranging from 40 to 80 mm, as well as the locomotion challenge rendered difficult by the device having to successfully negotiate the colonic flexures. The design should also provide space to locate a camera at the distal end for visual inspection, introduction and use of instruments. When the size of a soft robot is scaled down inclusion of all these requirements in the design may become challenging because of the high air activation pressure or high activation voltage needed to propel the robot. In addition, the design has to meet stringent medical regulatory standards. Micro and nano robots, developed during the last decade, have lacked therapeutic and diagnostic functionalities. Thus, several challenges have to be resolved before such robots can be realised for medical use.

Soft robots can be actuated by shape memory alloys, cables and air, external magnetic field, light, or by a combination of different actuation systems. Nature is inspirational for such designs and inchworm locomotion has led to several reported studies together with anchoring methods included in the design to increase the contact force and to address the direction of the locomotion. Toroidal balloons have been proposed to anchor the inchworm device inside a rigid tube or colon. However, most of the previous balloon inchworm-like locomotion designs rely on a linear actuator with only one degree of freedom (DOF)—for forwards and backwards travel—and which can be bent passively by pushing against the luminal internal wall to proceed around corners. This, however, may not be possible when the device has to negotiate acute corners or exceed high force against the inner wall of a lumen to achieve passive bending.

Accordingly, in a first aspect of the present invention, there is provided a robot for endoscopy comprising:

a leading balloon and a trailing balloon, each balloon being configured for selective inflation to anchor a portion of the robot within a pipe;

a soft-pneumatic actuator located between the leading balloon and the trailing balloon and which is operable to extend or contract the robot in a longitudinal direction; and

a cavity extending through the trailing balloon, actuator and leading balloon for provision of cabling and/or instruments; and

wherein the actuator comprises a plurality of chambers arranged such that movement of the leading balloon in any direction in a 360 degree circumference around the longitudinal direction is effected by pneumatic control of fluid within the plurality of chambers.

Thus, embodiments of the invention comprise a soft robot which can be formed through a simple construction process as it does not require complex rigid mechanical parts or internal electronics such as sensors, thus minimising production time and cost and consequently, making the robot disposable. This represents a significant advantage for medical applications because cleaning and sterilisation is no longer required. The design advantageously provides a robot having high dexterity and active control to facilitate smooth and safe locomotion in a tortuous path such as that of a colon. In particular, each of the leading and trailing balloons can be considered to provide 1 degree of freedom (DOF) relating to its inflation and deflation—while the actuator provides 3 degrees of freedom (DOF)—pitch, yaw and forward/backward. Furthermore, the hollow cavity facilitates cabling (such as for fluid control of the balloons or actuator) and location of instruments such as a camera in the front section of the robot.

Notably, the soft pneumatic actuator does not require any external force and can locomote by internal forces alone via fluid control in the plurality of chambers.

The leading balloon and/or trailing balloon may have a shape which is toroidal or part-toroidal. For example, the leading balloon and/or trailing balloon may have a shape comprising at least an exterior portion of a toroid. In some embodiments, the shape comprises at least an exterior half of a toroid. In some embodiments the shape comprises more than an exterior half of a toroid.

The cavity may be substantially cylindrical.

The plurality of chambers may be constrained in a lateral direction such that the pneumatic control effects expansion or contraction of each chamber in a longitudinal direction.

The actuator may be a positive differential pressure (PDP) actuator.

The actuator may comprise three or four chambers.

Each one of the plurality of chambers may be arranged for expansion in substantially the longitudinal direction to push a common face plate to direct the robot.

The robot may further comprise a closed-loop control system arranged to monitor fluid volume and/or pressure in the leading balloon and/or trailing balloon to determine when to stop inflation. The monitoring may be performed externally of the robot itself (i.e. via an external control apparatus monitoring fluid feedback) so as to avoid the need for monitoring components or sensors within the robot itself. In this way, the leading and/or trailing balloons may be configured to provide an adjustable diameter when inflated such that they can serve to anchor the robot in pipes with different internal diameters. Accordingly, a single device may be employed in a number of different applications such that a large inventory of devices or balloons is not required whilst still ensuring safe interaction of the balloon with the pipe walls.

None of the prior art devices has an adjustable diameter of balloon or an external control system arranged to facilitate this and therefore they can only work for a specific pipe diameter. Such limited variation of the external balloon diameter reduces the anchorage force possible in the various regions of the colon. However, this is overcome with embodiments of the invention.

The robot may be configured as a disposable device. Thus, the robot may be configured such that electronic components are all external or easily removable (e.g. in the case of a camera provided in the cavity).

The leading balloon and/or trailing balloon may comprise a soft silicon rubber.

The leading balloon and/or trailing balloon may have a deflated external diameter in a range of 12 mm to 20 mm.

The leading balloon and/or trailing balloon may have an inflated external diameter of up to at least 90 mm.

The leading balloon and/or trailing balloon may have an activation pressure of 100 kPa or below, 50 kPa or below or 10 kPa or below. In some embodiments, the leading balloon and/or trailing balloon may have an activation pressure of 6.1 kPa or 2.5 kPa.

The cavity may have a diameter in a range of 1 mm to 8 mm.

The actuator may have an external diameter in a range of 12 mm to 20 mm.

The chambers may each have a length in a range of 12 mm to 30 mm.

The robot may further comprise fluid control tubes fluidly connected to each of the leading balloon, trailing balloon and plurality of chambers.

The robot may comprise a camera or other electronic components in the cavity.

### BRIEF DESCRIPTION OF THE DRAWINGS

Non-limiting embodiments of the invention will now be described for the sake of example only, with reference to the following drawings in which:

FIG. 1 shows schematically the locomotion and degrees of freedom of a robot in accordance with a first embodiment of the invention;

FIG. 2 shows a prototype of the robot of FIG. 1 in a) a rest configuration with both balloons deflated, b) a curved configuration and c) a rest configuration with both balloons inflated;

FIG. 3 shows the robot of FIG. 2 in a) an end perspective view similar to FIG. 2c); b) a longitudinal cross-sectional view through the image of FIG. 3a); and c) the steps of locomotion of the robot of FIG. 3a);

FIG. 4 shows a schematic diagram of a control system for the actuator shown in FIG. 3;

FIG. 5 shows experimental results of the robot of FIG. 3 relating to the expansion of a balloon and its associated anchorage force;

FIG. 6 shows schematic views of the actuator of FIG. 3 and experimental results relating to the bending and extension of the actuator;

FIG. 7 shows experimental results of the robot of FIG. 3 relating to an actuation force for active extension and passive contraction of the actuator;

FIG. 8 illustrates steps for construction of the actuator of FIG. 3;

FIG. 9 illustrates steps for constructions of the balloons of FIG. 3;

FIG. 10 illustrates experiments of the robot of FIG. 3 in a) a vertical tube of varying internal diameter and b) a mock plastic colon; and

FIG. 11 is a more detailed view of the mock plastic colon of FIG. 10b).

### DETAILED DESCRIPTION OF THE EMBODIMENT

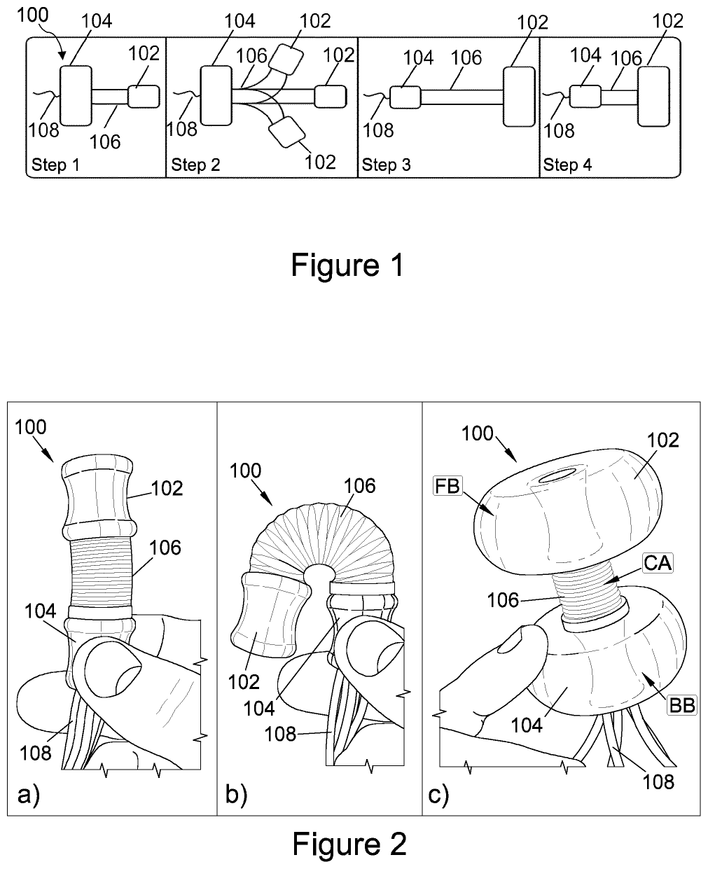

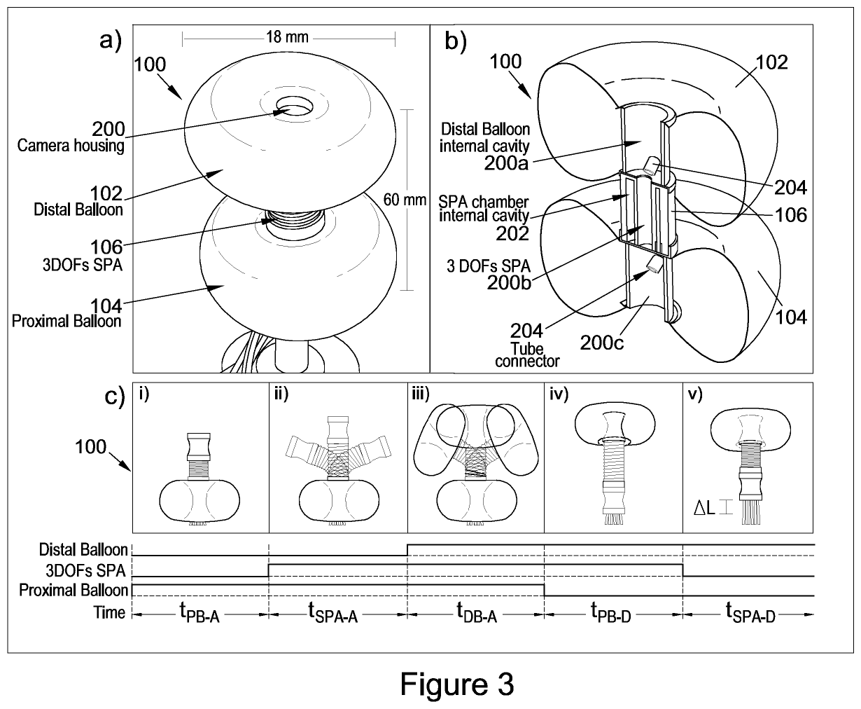

FIG. 1 shows schematically the locomotion and degrees of freedom of a robot 100 configured for endoscopy (particularly, colonoscopy) in accordance with a first embodiment of the invention. The robot 100 comprises a leading generally toroidal balloon 102 and a trailing generally toroidal balloon 104, each balloon 102, 104 being configured for selective inflation to anchor a portion of the robot 100 within a pipe (not shown). A cylindrical soft-pneumatic actuator (SPA) 106 is located between the leading balloon 102 and the trailing balloon 104 which is operable to extend or contract the robot 100 in a longitudinal direction. A cylindrical cavity (shown most clearly in FIG. 3) extends through the trailing balloon 104, actuator 106 and leading balloon 102 for provision of cabling and/or instruments (not shown).

As also shown in FIG. 3, the actuator 106 comprises a plurality of chambers (in this case three) arranged such that movement of the leading balloon 102 in any direction in a 360 degree circumference around the longitudinal direction is effected by pneumatic control of fluid within the chambers.

A tether 108 extends from the trailing balloon 104 to maintain contact with the robot 100 in use and to supply fluid to control the direction and operation of the robot 100. More specifically, the tether 108 comprises a bundle of five fluid control lines for supplying fluid to the leading balloon 102, the trailing balloon 104, and each of the chambers in the actuator 104.

The robot 100 may be referred to as a Soft Pneumatic Inchworm Double balloon (SPID) robot. An advantage of using balloons in the present robot 100 is that they allow for the possibility of increasing a contact force to the pipe wall to anchor the robot 100, despite the light weight of the robot 100.

Each of the leading balloon 102 and trailing balloon 104 have 1 degree of freedom (DOF)—inflation and deflation—while the actuator 106 can extend and contract (1 DOF) and rotate around 2 axes (2 DOF)—pitch and yaw. Accordingly, the robot 100 has 5 DOF in total and can be actively controlled to move forwards or backwards with enough dexterity to go around narrow acute corners, including colonic flexures.

The robot 100 is configured for locomotion via the following four steps as shown in FIG. 1:
1^{st} step: the trailing balloon 104 is inflated to anchor the robot 100 to the surrounding cavity;
2^{nd} step: the actuator 106 extends forwardly and is bent if required to follow the shape of the cavity to be inspected;
3^{rd} step: the leading balloon 102 is inflated to anchor the robot 100 at a new position and the trailing balloon 104 is deflated to release the anchor on the rear portion of the robot 100;
4^{th} step: the actuator 100 is contracted to draw the trailing balloon 104 forward and thereby propel the robot 100 to a new forwardly position.

The robot 100 can therefore locomote in a tortuous environment along any combination of the three dimensions by using the balloons 102, 104 as anchors in combination with the dexterous actuator 106.

FIG. 2 shows a prototype of the robot 100 of FIG. 1 in a) a rest configuration (i.e. whereby the actuator 106 is not activated) with both balloons 102, 104 deflated; b) a curved configuration whereby the actuator 106 is activated so as to loop the leading balloon 102 around 180 degrees to be positioned adjacent the trailing balloon 104; and c) a rest configuration similar to FIG. 1(a) but wherein both balloons 102, 104 are inflated.

The robot 100 shown in FIG. 2 has an external diameter of 18 mm. Each of the leading balloon 102 and trailing balloon 104 can expand more than 90 mm in diameter. The actuator 106 has a diameter of 14 mm and a length in the rest position of 23 mm but which is extendable on activation up to 75 mm. The actuator 106 has a high dexterity to achieve rotation along both X and Y axes. Tests have been performed using manual syringes to inflate the balloons 102, 104 and chambers in the actuator 106 for the desired locomotion, as will be described in more detail below. However, in a commercial device it is envisaged that a smart control system will be employed to increase the locomotion speed.

FIG. 3 shows the robot 100 of FIG. 2 in a) an end perspective view similar to FIG. 2(c); b) a longitudinal cross-sectional view through the image of FIG. 3(a); and c) the steps of locomotion of the robot 100 of FIG. 3(a).

More specifically, FIG. 3(a) shows a perspective view of the robot 100 with the leading balloon 102 (also referred to as the distal balloon DB) and trailing balloon 104 (also referred to as the proximal balloon PB) inflated and an end of the central cavity 200 shown which in practice will house cabling and a camera for inspection and potentially other equipment or electronics for use during an endoscopic procedure.

The design parameters of robot 100, i.e., range of motion, external diameter, and overall length were chosen to conform to an average size and shape of a human colon. Thus, the robot 100 of the present embodiment has an external diameter (when at rest, i.e. uninflated) of 18 mm, a total length of 60 mm and a weight of 10 g. Both balloons 102, 104 have a cylindrical frame with an inner cavity of 13 mm in width and 20 mm in length. The tether 108 to activate the two balloons 102, 104 and the SPA 106 is composed of 5 silicon fluid control lines with an external diameter of 2.6 mm each for the SPA 106, and 3 mm each for the balloons 102, 104.

FIG. 3(b) shows a cross-sectional view of the robot 100 showing the cavity 200 extending through the centre of the robot 100 and being comprised of a distal cavity 200a surrounded by the distal balloon 102, an actuator (SPA) cavity 200b in the centre of the actuator 106 and a proximal cavity 200c surrounded by the proximal balloon 102. Notably, the actuator cavity 200b has a smaller diameter than the distal cavity 200a and the proximal cavity 200c. This is to allow space for three longitudinal fluid chambers 202 evenly distributed around the actuator cavity 200b for activation and bending of the robot 100 as will be explained in more detail below. Tube connectors 204 are also shown for fluidly coupling a fluid control line (not shown) to each of the distal and proximal balloons 102, 104 for inflation and deflation thereof. As clearly shown in FIG. 3(b), the distal and proximal balloons 102, 104 are substantially toroidal in shape surrounding the cavity 200.

FIG. 3(c) shows steps of the bio-inspired locomotion of the robot 100, similar to FIG. 1. However, in this case a timeline is shown alongside each step such that t_{PB-A} A is the time needed to activate the proximal balloon 104 in step i); t_{SPA-A} is the time required to activate the SPA 106 in line with the orientation of the colonic lumen in step ii); t_{DB-A} is the activation time of the distal balloon 102 to provide anchorage in step iii); t_{PB-D} is the deactivation time of the proximal balloon 104 in step iv); and t_{SPA-D} is the deactivation time of the SPA 106 to move the proximal balloon 104 forward in step v).

The present embodiment relies on a positive differential pressure (PDP) rather than a buckling actuator, which relies on negative differential pressure (NDP). PDP actuators can provide a positive force to extend and push the distal balloon 102 forward. This represents a considerable advantage for a colonoscopic robot as it enables the device to open a collapsed colonic lumen. An active pushing force improves the ability of the robot 100 to negotiate around corners adapting its shape by its intrinsic flexibility to conform to the contours of the flexure. The realisation of a PDP actuator 106 as described above with 3 DOF imparts a further advantage by being considerably smaller than a NDP because of its simpler manufacturing process. When deactivated, a PDP actuator relies on a passive force provided by the mechanical properties of the elastomeric body as it contracts and pulls forward the proximal balloon 104. A key limitation in using a NDP actuator is in the manufacture of an actuator with 3 DOF plus an inner cavity 200b which is of a suitably small and compact design. Because of the established advantages of a PDP as compared to a NDP actuator, the proposed robot 100 includes a PDP soft pneumatic actuator (SPA) 106.

In the present embodiment, the SPA 106 is composed of 3 longitudinal chambers 202 and a central 5 mm diameter circular cavity 200b, with a cross-sectional area of 2.6 mm². The external diameter is 18 mm and the SPA 106 has a total length of 20 mm. Several length to diameter ratios have been tested and the one selected for the present robot 100 exhibited a good compromise between lateral bending capability enabling high dexterity, and longitudinal elongation to enhance locomotion speed. Higher dexterity could be achieved by using four chambers 202 although a further fluid control line would be required for this which would increase the diameter of the tether 108 and hence increase the drag force with inevitable reduction of locomotion speed.

The balloons 102, 104 in the present embodiment are made of hyper elastic materials to allow for an increase in the inflated balloon diameter to ensure it can serve as an anchor in pipes or tubes having a range of different diameters. The desired range is input into the design of the balloon, to define its thickness and required internal fluid pressure. For applications where a high activation pressure can be problematic (e.g. medical applications), a reduced balloon wall thickness can reduce the maximal pressure required for inflation thereby making the robot 100 safe in case of rupture.

The balloon contact with the external environment is uniformly distributed and related to the internal fluid pressure. Advantageously, an increase in contact friction can be achieved by an increase in fluid pressure instead of an increase in robot 100 weight. This is particularly useful because the robot 100 can combine a light weight and a high friction force. By way of background, friction (F) in an object is related to its weight (W) through F=W·μ, where μ is the coefficient of friction between the material and the external surface. With a balloon, the friction is also related to the contact pressure (P), as described by the following equation F=(P+W)·μ. Additional anchoring forces can therefore be produced if the external environment can be expanded. The balloon will gently adapt its shape to the deformed wall of the pipe or colon and the mechanical interaction will increase the anchor forces.

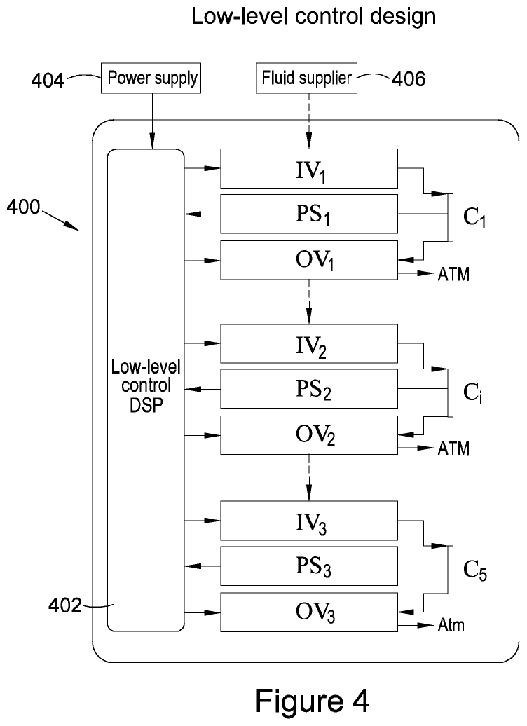

FIG. 4 shows a schematic diagram of an external control system 400 for the actuator 106 shown in FIG. 3. The control system 400 comprises a low level control digital signal processor (DSP) 402 connected to a power supply 404 and which is configured to control inlet valves (IVᵢ) and outlet valves (OVᵢ) between a fluid supply 406 and each chamber (i) 202 in the actuator 106. In operation, the fluid in each chamber 202 is controlled using the inlet valve (IVᵢ), outlet valve (OVᵢ) and a pressure sensor (PSᵢ) and each proportional valve (IVᵢ and OVᵢ) is controlled by adjusting a duty cycle of a pulse width modulation (PWMᵢ) output provided by the DSP. The pressure sensor (PSᵢ) monitors the chamber pressure and is used as a reference variable for a closed-loop control.

It should be noted that the fluid employed in embodiments of the invention may be air, water or another gas or liquid.

A series of experiments were performed by manually controlling the activation of the distal and proximal balloons 102, 104, together with the SPA 106. The activation was performed using piston-cylinders coupled to fluid control lines to control a volume of air supplied to the balloons or chambers of the actuator 106. The piston-cylinder used to activate each balloon had a volume of 100 mL, whereas the 3 piston-cylinders used to activate the SPA 106 had a volume of 10 mL each. The robot 100 and the fluid control lines were lubricated with Vaseline™ to reduce any friction during locomotion.

It is well established that one of the main challenges for a robotic colonoscopy consists of the ability of the robot 100 to negotiate the acute angled splenic flexure at the junction between the transverse and descending colon. This flexure has an angle ranging between 40 degrees and 50 degrees and a diameter of 30 mm after CO₂ inflation of the colon.

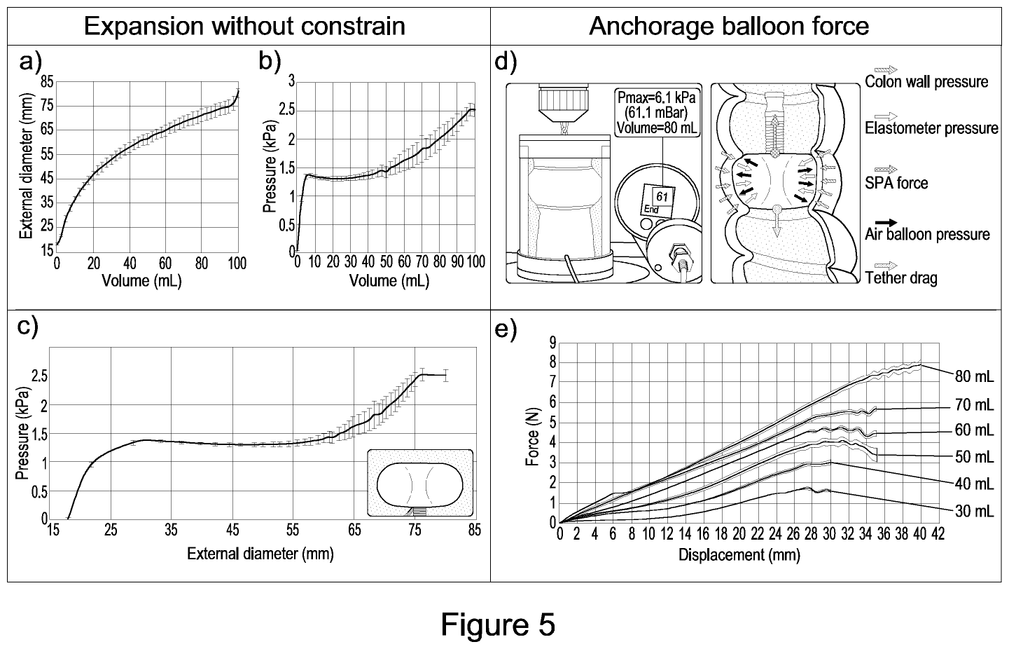

The two balloons 102, 104 in the present embodiment of the robot 100 are made from Ecoflex™ 00-30 and can be activated with a low air pressure ensuring low pressure is exerted on the colonic wall. The experiments were performed in a quasi-static configuration activating each balloon with air controlled manually by using an external piston-cylinder and measuring the inflated volume and air pressure. Each experiment was performed five times with mean value and standard deviation shown in the graph of FIG. 5.

More specifically, FIG. 5(a) shows the experimental results of the balloon with unconstrained radial expansion reporting air volume vs. balloon external diameter. FIG. 5(b) shows the results of air volume vs. activation air pressure and FIG. 5(c) shows the results of external balloon diameter vs. activation air pressure. FIG. 5(d) shows the static forces and pressures on the balloon inside the colon wall which result from the inchworm locomotion described above. FIG. 5(e) shows anchorage force which increases with air volume.

Each balloon 102, 104 has an external diameter at rest of 18 mm and when activated it expands above 80 mm (see FIG. 5(a)) with an activation pressure below 2.5 kPa without expansion constrain (see FIG. 5(b)). The air volume vs. external diameter exhibits a monotonic function, resulting in an air volume feedback reference for the implementation of a closed-loop control such as one which controls a piston stroke in a pneumatic cylinder. The air volume vs. pressure graph exhibits three distinct phases. In phase one, as the air volume rises from 0 to 10 mL, the pressure increases rapidly to 1.4 kPa with a negligible increase of the balloon external diameter. In phase two, as the air volume rises from 10 to 50 mL, the pressure decreases slightly with its value about 1.4 kPa. In this phase, the balloon diameter starts to increase. Phase three is when the air volume exceeds 50 mL and the pressure increases with a linear fashion up to 2.5 kPa.

The graph reported in FIG. 5(c) is obtained by combining the two graphs from FIGS. 5(a) and 5(b) to relate the external diameter of the balloon with the internal air pressure. This graph shows a non-monotonic function. This implies that there is no constant relation between a given diameter and a given pressure. This behaviour is explained by the fact that the internal air pressure increases the wall tension as the diameter increases (Laplace's law) in accordance with the mechanical properties of the balloon material.

The characteristics of this balloon therefore require that, to adjust its diameter, a closed-loop control system has to use the air volume as a feedback-reference input, because a given balloon external diameter cannot be controlled by using the air pressure. A wide inflated balloon diameter allows the robot 100 to secure a stable anchorage inside the colon between haustra providing sufficient force to move propel the robot 100 forward. The anchorage force was measured by using an Instron® 5564 dual column with the load cell connected to the balloon as shown in FIG. 5(d) and by pulling the load up at a constant speed of 1 mm/s. The displacement vs. force profile for each volume of air inflated in the balloon is shown in FIG. 5(e). The maximal anchorage force was 7.9 N with a balloon activation air volume of 80 mL and a pressure of 6.1 kPa. The force was tested by using a vertical tube made from transparent thin plastic film (15 mm thickness) with the balloon being anchored between 2 elastic bands simulating a haustral fold.

Previous studies proposed balloon designs with a limited range in expansion of the external diameter, and lack of internal space to accommodate a camera, despite the fact that this requirement is essential for the design of a robot for colonoscopy. A large range in the external diameter (on inflation), allows the robot 100 to be securely anchored in the different sections of the colonic wall. An internal cavity 200 may be used for accommodation of electronic components for control (such as an inertial measurement unit (IMU) or camera). Both these requirements have been addressed in the design of the current balloons 102, 104.

The balloon structure in the present embodiment includes a 3D printed cylindrical frame made of Vero-Clear surrounded by a thin layer (thickness of 0.6 mm) of silicon rubber Ecoflex™ 00-30. Compared to other silicon rubbers, Ecoflex™ 00-30 exhibits lower shore hardness and higher elongation break. This results in a lower activation pressure and higher compliant behaviour, essential to reduce the force applied to the colonic wall and consequently increasing patient compliance. A higher stiffness elastomer would require higher activation pressure, which may be problematic in case of balloon rupture. The tube connector 204 is located in an inner part of the frame for the balloon activation.

The balloon, when activated, adapts its shape to the colonic haustral fold producing an anchorage force (FA) essential for the inchworm locomotion. This force has 2 major components: Coulomb friction (FC) and marginal resistance (FM). The Coulomb friction is related to the force of the balloon against the colonic wall (FB) and the coefficient of friction m. This force includes also the weight of the robot (FR) although, because of its light weight (10 g), this force is negligible compared to FB. The FC can be low because of the slippery colonic mucosa surface. The marginal resistance is related to the longitudinal deformation of the colonic wall.

During a colonoscopy, air or gas is used to inflate and expand the colonic lumen with an internal pressure up to 7.6 kPa, which is higher than the balloon activation pressure required by the robot 100 of the present embodiment. The pressure of the balloon against the colonic wall is the result of the internal air pressure (PB) minus the elastomer mechanical pressure (PE). This produces an external pressure on the colon wall, which is much lower than the pain pressure threshold encountered during standard colonoscopy.

In some embodiments, internal air volume and/or pressure may be monitored such that when it reaches a predetermined threshold inflation stops automatically.

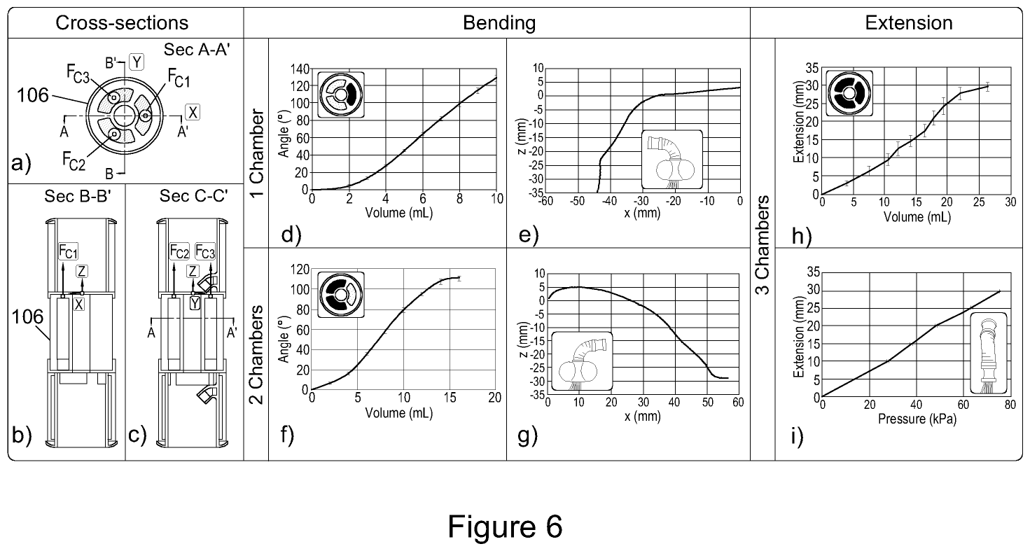

Free displacement studies of the robot 100 of the present embodiment were performed by activating the SPA 106. As shown in FIG. 6(a) the SPA 106 includes 3 chambers (C1;C2;C3 and their forces FC1;FC2;FC3) symmetrically disposed around the central cavity as shown in the cross-section A-A′ of the SPA illustrated in FIG. 6(c). FIG. 6(b) shows the cross-section B-B′ illustrated in FIG. 6(a) and the force produced by activation of chamber C1 for positive bending. FIG. 6(c) shows the cross-section C-C′ and the force produced by the activation of chambers C2 and C3 for negative bending.

Experiments of the positive bending around the Y axis are shown in FIG. 6(d) with a maximal value of 130 degrees. The activation of the chamber C1 moves the distal part of the robot 100 in the X-Y plane, with the trajectory shown in FIG. 6(e). Experiments of the negative bending around the Y axis are shown in FIG. 6(f) with a maximal value of 110 degrees. The trajectory of the bending in the plane X-Y is shown in FIG. 6(g).

FIGS. 6(d) and (f) show similar bending trajectories. However, the higher force generated by the simultaneous activation of two chambers during the positive bending produces an X displacement wider than the negative bending, when only one chamber is activated.

The extension along the Z axis is obtained by the simultaneous activation of all three chambers C1;C2;C3 producing a maximal extension of 30 mm as shown in FIG. 6(h). The SPA volume vs. pressure monotonic is shown in FIG. 6(i). This monotonic behaviour is different from the non-monotonic balloon profile, and is due to the anisotropic structure of the SPA 106 wherein the wall is reinforced with cotton threads to constrain lateral expansion. Details of the construction process for this particular SPA 106 are provided below. Notably, during activation, the cross-sectional area of each chamber remains almost constant generating a forwards force proportional to the internal air pressure.

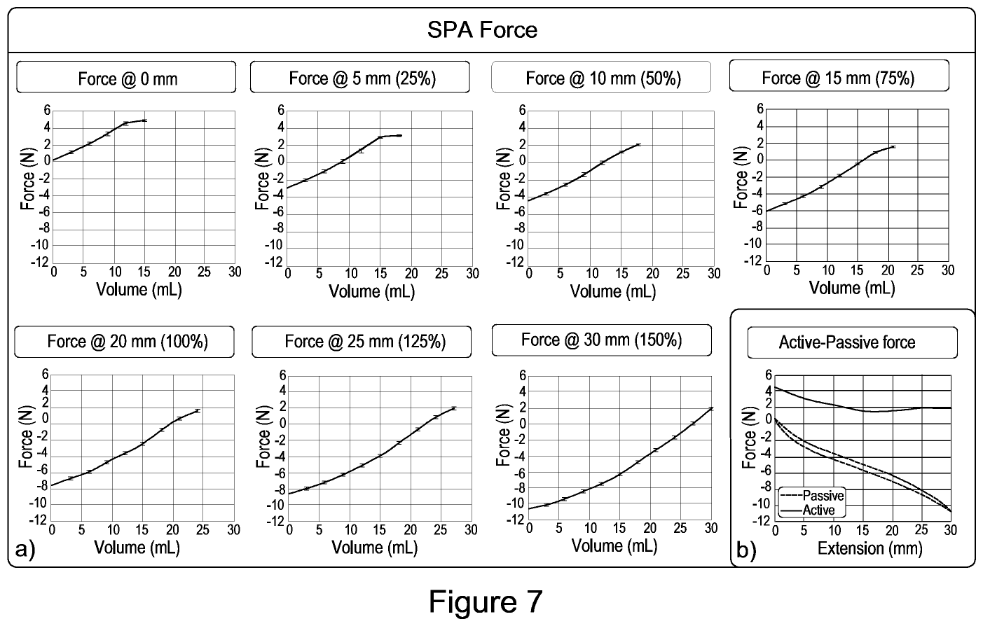

FIG. 7 shows experimental results of the robot 100 relating to an actuation (or blocking) force for active extension and passive contraction of the actuator 106. In particular, FIG. 7(a) shows experiments of the SPA activation force with its extension up to 30 mm and FIG. 7(b) shows the force produced when the SPA is activated and when deactivated.

These experiments were performed by using an Instron® 5564 column connected to the tip of the SPA 106. The three chambers were activated simultaneously by controlling the volume of air with three piston-cylinders, with an internal volume of 10 mL. Each experiment was performed five times in quasi-static configuration. The force produced by the SPA when not activated was investigated by measuring the stress strain graphs using the Instron® 5564 dual column connected to the tip of the SPA. The speed at which the test was run was 1 mm/s with a maximal stroke of 30 mm (150% of the original length of the SPA 106).

The graphs in FIG. 7(a) show volume vs. force in different extension from 0 up to 30 mm with a maximal pushing longitudinal force of 4.8 N. This force is needed to advance the robot 100 forward and open a path in case of luminal collapse. The output force decreases with the extension due to the increase of the resistance of the polymer when it is stretched.

FIG. 7(b) shows the extension vs. force profile when the SPA is activated (blue line), and the passive force produced by the polymer mechanical resistance when the SPA is deactivated (red line). The active force decreases linearly from 0 to 15 mm and then retains a value around 2 N. The passive force has an almost linear profile with a negligible hysteresis due to the mechanical properties of the polymer. The passive force is required to pull the tether and overcome its drag during locomotion. These experiments confirm the high dexterity of the SPA 106 and the high force vs. weight ratio.

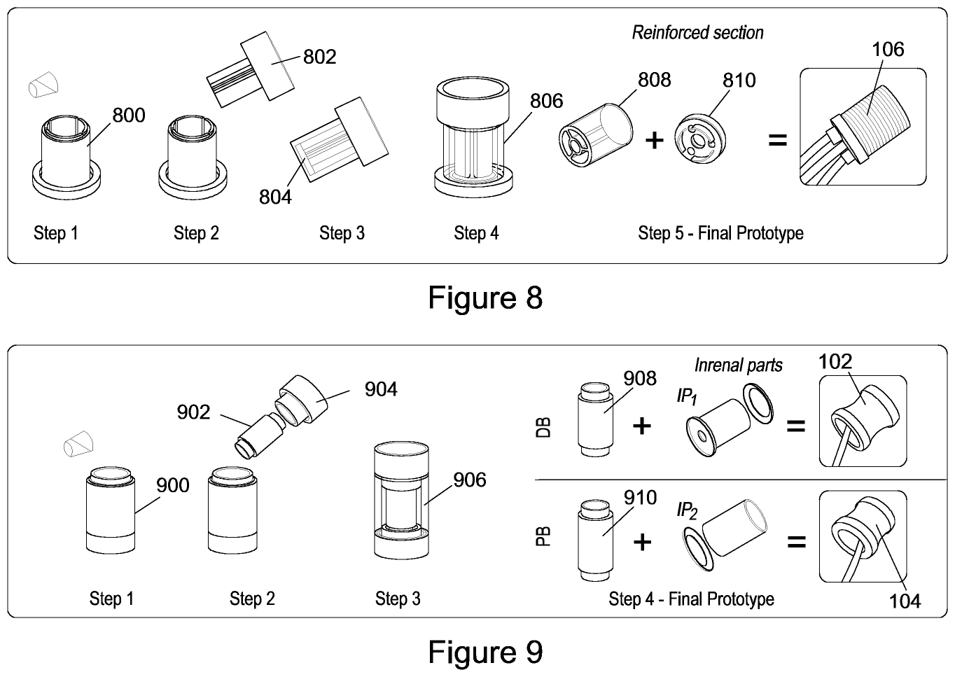

FIGS. 8 and 9 illustrate steps for construction of the actuator 106 and balloons 102, 104, respectively, for the robot 100 in accordance with an embodiment of the invention. Each of these components is constructed and tested separately before being assembled and glued together to form the robot 100.

The construction process of the SPA 106 comprises five steps as depicted in FIG. 8. To achieve an anisotropic behaviour of the SPA 106 for avoidance of lateral expansion during inflation, the outer wall comprises Ecoflex™ 00-30 reinforced with cotton threads. All the internal walls of the chambers 202 are also made of Ecoflex™ 00-30. Three silicon tubes (outer and inner diameter of 2.6 mm and 1.6 mm respectively) are connected to the base of the SPA 106 and used for activation. The soft polymer (SP) is a composite of Ecoflex™ 00-30 part A and B mixed in 1:1 ratio.

The steps carried out to construct the SPA 106 are follows:
Step 1) bottom section 800 of the moulder is assembled and the SP is poured in and degassed by vacuuming at 0 atm for 10 minutes;
Step 2) insertion of the top section 802 of the moulder followed by curing at 100 degrees for 30 minutes;
Step 3) bottom section 800 of the moulder is removed, while the top section 802 is kept inside the cast 804 to keep it rigid. Threads are wrapped around the cast 804 with a gap of 1 mm to constrain lateral expansion;
Step 4) bottom section and additional external parts of the mould 806 are assembled, and the SP is poured to form an external layer to protect and fix the cotton threads, forming an anisotropic polymer structure. The cast is again cured at 100 degrees for 30 minutes;
Step 5) the cast 808 is removed and a bottom reinforced section 810 made of VeroClear-RGB10 polymer together with three silicon tubes are glued together using Sil-Poxy® Silicone Adhesive to form the actuator 106.

Finally, the actuator 106 undergoes a quality check comprising a visual inspection and air leakage testing.

The construction process of the two balloons 102, 104 is similar and includes an internal part made of VeroClear-RGB10 polymer and an external thin layer of Ecoflex™ 00-30. The internal 3D printed parts are shown in FIG. 9, where IP₁ (internal part 1) relates to the distal balloon 102, and IP₂ (internal part 2) relates to the proximal balloon 104. The internal part has an internal silicon tube connector 204 for the balloon activation.

The steps needed to construct the balloons 102, 104 are as follows:
Step 1) bottom section 900 of the moulder is assembled and the SP is poured in and degassed by vacuuming at 0 atm for 10 minutes;
Step 2) insertion of the middle section 902 and top section 904 of the moulder;
Step 3) cast 906 is cured at 100 degrees for 30 minutes and then removed from the mould;
Step 4) top and bottom lips of the casts 908, 910 are folded and glued on top of the IP₁ and IP₂ using Sil-Poxy® Silicone Adhesive and left to cure for at least 1 hour.

When both balloons 102, 104 are constructed, visual inspection and air leakage testing is used for quality assessment of the final product.

The silicon tube for the activation of the distal balloon 102 is inserted inside the SPA 106 after lubrication (Vaseline) to reduce friction in the internal cavity 200 during locomotion. Then the three tubes used for the activation of the SPA 106 are inserted through the proximal balloon 104 and the silicon tube for the activation of the proximal balloon 104 is connected. Next, the SPA 106 is glued to the distal and proximal balloons 102, 104 using Sil-Poxy® Silicone Adhesive and left to cure.

Experiments were performed in a rigid tube of different diameters and in a plastic colonic phantom to investigate locomotion speed and corner negotiation, anchorage and push/pull forces.

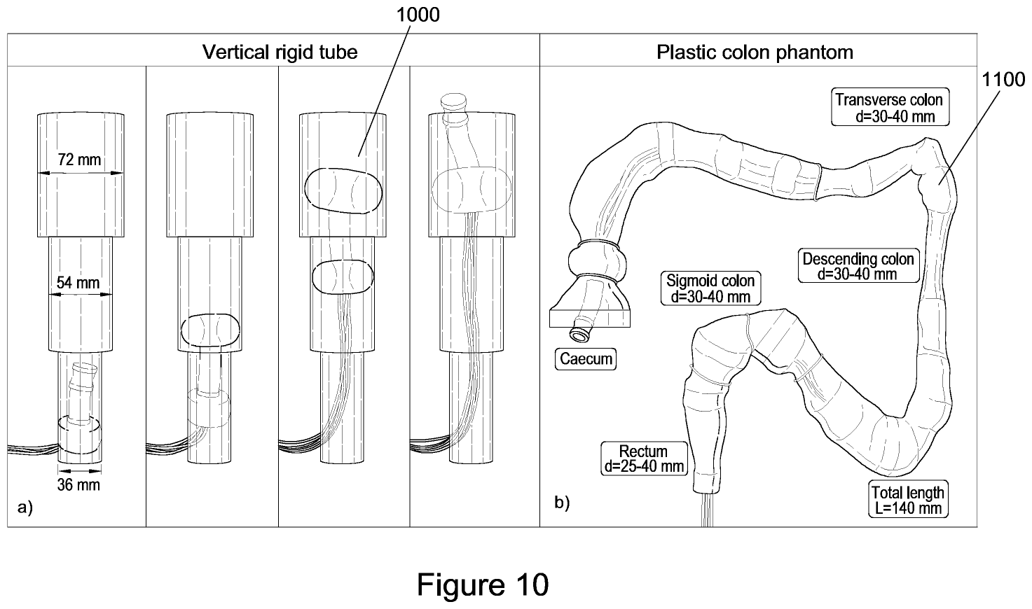

FIG. 10(a) shows the ability of the two balloons to adapt to different diameters in different sections of a rigid vertical tube 1000. FIG. 10(b) shows the robot 100 in different sections of a plastic colon 1100.

Vertical locomotion and the robot's capability to adapt to the shape of different colonic sections were tested in a rigid vertical tube 1000 with internal diameters of 36 mm, 54 mm, and 72 mm, respectively, each with a length of 100 mm, providing a total of 300 mm, as shown in FIG. 10(a). This experiment confirmed the ability of the balloons 102, 104 to conform to the shape of different colonic diameters.

A colonic phantom 1100 was designed and constructed to test the locomotion performance of the robot 100 based on the size and configuration of the average human colon anatomy.

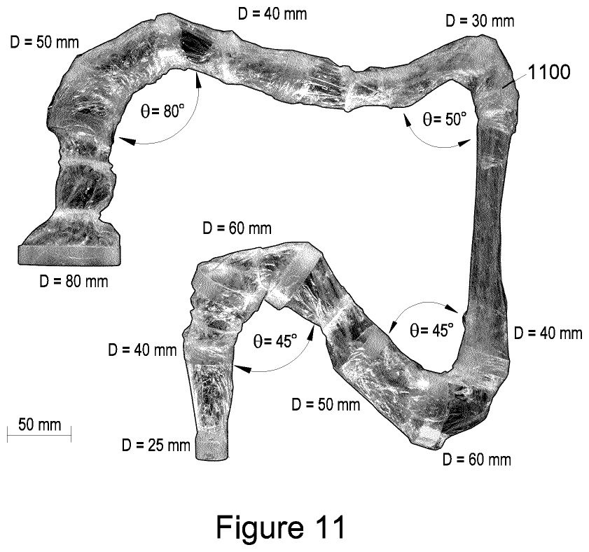

FIG. 11 is a more detailed view of the mock plastic colon of FIG. 10(b). The total length was 1.4 m, internal diameters ranged from 25 mm up to 80 mm, and flexure angles were from 45 degrees to 80 degrees. The design of the colonic phantom 1100 was made by using fifteen 3D printed support rings. A transparent thin plastic film (15 mm thickness) was wrapped around each couple of consecutive rings with lubricant inside to minimise friction. Elastic bands were fixed externally to the plastic film by using Sil-PoxyR Silicone Adhesive to avoid movements.

This set up therefore addressed the locomotion in a more challenging environment including the 3D path of a colon as well as a thin and weak plastic wall. Lubricant in the form of Vaseline™ was applied between the robot 100 and the wall made of transparent thin plastic film to reduce the locomotion friction. The elastic bands were used to simulate haustral folds in the colon.

The robot 100 showed high dexterity in negotiating the narrow corners with good locomotion speed. The experiments were performed in accordance with a standard colonoscopy procedure. Hence, in an initial phase of the colonoscopy procedure, the endoscopist is focused on reaching the caecum. Thereafter, inspection is undertaken during the second stage as the endoscope is withdrawn slowly. The robot 100 experiments reproduced the same procedure, showing that the robot 100 was able to reach the caecum in 8 minutes 30 seconds, covering a total length of 1.4 m at an average speed of 2.8 mm/s. After caecal intubation, the robot 100 was withdrawn by traction on the air tube tether. This phase took about 1 minute with an average speed of 25 mm/s although in a real scenario this may take longer for a precise evaluation of the colonic mucosa. During this phase the robot 100 could be activated, enabling forward locomotion for subsequent inspection when considered necessary to ensure that no abnormality was missed. The balloons, when anchored, allow precise control and steering of the robot tip for inspection and/or treatment.

The robot 100 therefore tested successfully in the deformable plastic colon phantom 1100 of similar shape and dimensions to the human anatomy, exhibiting efficient locomotion by its ability to deform and negotiate flexures and bends.

Embodiments of the invention relate to a soft pneumatic inchworm double balloon robot 100 configured for colonoscopy. The robot 100 is made of 3D printed components and Ecoflex™ 00-30 with high compliance and flexibility for intrinsic locomotion demonstrated by experiments in a plastic deformable colon phantom 1100. The dexterous and compliant behaviour of the 3 DOF soft pneumatic actuator 106 connecting the 2 balloons 102, 104 enables the robot 100 to negotiate acute corners and the highly compliant double balloon structure readily adapts to conform to the shape of the various colonic sections and their diameters. Construction from soft materials carries several physical advantages including innate flexibility, gentle atraumatic contact with the colonic wall and low production cost, essential for production of a disposable device. This can negate issues concerning sterilisation, cross contamination and maintenance. The low Young's modulus provides a passive compliant interaction with the colonic wall reducing pressure for anchorage. During the experiments, the balloons never sustained any air leakage, essential for assuring the device reliability. A camera is needed for colon inspection, which can be mounted in the cavity of the distal balloon. Traditional biopsy instruments can be used both for diagnostic and therapeutic functions. The robot 100 can incorporate a tube extending from the camera to an external console for insertion of instruments for treatment of suspect lesions.

The discussed experiments primarily concerned the locomotion design and its transit rate in the plastic colon phantom 1100. The insertion of the tether through the anus will produce additional friction and drag force. However, this friction can be almost abolished by use of an external device to feed the tether during the locomotion or with the use of a dedicated access port. The activation of the SPA chambers and the balloons was obtained by using manual controlled external piston-cylinders, successfully demonstrating its functionalities. An active control will improve the locomotion speed, manoeuvrability, and will ease the procedure by reducing activation and deactivation times. Such control can be implemented via an external user console with a joystick or by using a smart-control to follow autonomously the colonic lumen. This should ensure very precise control of the robot 100 as well as assistance in the training or use of the robot 100 by a technician or nurse practitioner. In such scenarios, a trained colonoscopist would potentially supervise several simultaneous ongoing procedures acting, e.g. requesting biopsy or reverse locomotion to obtain a second look, with a disruptive reduction of cost. Finally, by reducing the pressure against the colon wall, the robot 100 may contribute to increase the compliance rate for CRC screening of the asymptomatic population, towards mass screening campaigns for early diagnosis.

The design of a smart robot for colonoscopy is challenging because of the limited available space, slippery internal surfaces, and tortuous 3D shape of the human colon. Locomotion forces applied by an endoscopic robot may damage the colonic wall and/or cause pain and discomfort to patients. Embodiments of the present invention provide a robot in the form of a Soft Pneumatic Inchworm Double balloon (SPID) device which is suitable for colonoscopy amongst other things. The balloons provide anchorage into the colonic wall for a bio-inspired inchworm locomotion. The proposed design reduces the pressure applied to the colonic wall and consequently reduces pain and discomfort during the procedure. The robot is made of soft elastomeric material and may be constructed from 3D printed components, hence with low production costs essential for a disposable device.

Some advantages of embodiments of the invention when compared to previous reported devices are i) high dexterity in locomotion with 5 DOFs to overcome narrow bends and flexures, ii) a novel balloon design for improved anchorage of the robot in different sections whilst exerting a low external pressure in the colonic wall, and iii) provision of adequate inner space for housing a camera, electronics, cables and tubes, as required for operation of the robot and endoscopic examination.

Whilst the foregoing description has described exemplary embodiments, it will be understood by those skilled in the art that many variations of the embodiments can be made within the scope of the present invention as defined by the claims. Moreover, features of one or more embodiments may be mixed and matched with features of one or more other embodiments.

### REFERENCES

[1] Wade Adams, Saivimal Sridar, Carly M. Thalman, Bryce Copenhaver, Hassan Elsaad, Panagiotis Polygerinos “Water Pipe Robot Utilizing Soft Inflatable Actuators”, Robosoft, 2018, Livorno, Italy
[2] Mohit S. Verma, Alar Ainla, Dian Yang, Daniel Harburg, George M. Whitesides, “A Soft Tube-Climbing Robot”, Vol. 5, n. 2, 2018, DOI: 10.1089/soro.2016.0078, SoftRobotics
[3] K. Wang, Y. Ge, and X. Jin, “A Micro Soft Robot Using Inner Air Transferring for Colonoscopy”, ROBIO, 2019, Shenzhen, China, 2013

1. A robot for endoscopy comprising:
a leading balloon and a trailing balloon, each balloon being configured for selective inflation to anchor a portion of the robot within a pipe;
a soft-pneumatic actuator located between the leading balloon and the trailing balloon and which is operable to extend or contract the robot in a longitudinal direction; and
a cavity extending through the trailing balloon, actuator and leading balloon, wherein the cavity is configured for provision of at least one of cabling or instruments; and
wherein the actuator comprises a plurality of chambers arranged such that movement of the leading balloon in any direction in a 360 degree circumference around the longitudinal direction is effected by pneumatic control of fluid within the plurality of chambers.

2. The robot of claim 1 wherein the plurality of chambers are constrained in a lateral direction such that said pneumatic control effects expansion or contraction of each chamber in a longitudinal direction.

3. The robot of claim 1 wherein the actuator is a positive differential pressure (PDP) actuator.

4. The robot of claim 1 wherein the actuator comprises three or four chambers.

5. The robot of claim 1 wherein each one of the plurality of chambers is arranged for expansion in substantially the longitudinal direction to push a common face plate to direct the robot.

6. The robot of claim 1 further comprising a closed-loop control system arranged to monitor at least one of fluid volume or pressure in at least one of the leading balloon or the trailing balloon to determine when to stop inflation.

7. The robot of claim 1 configured as a disposable device.

8. The robot of claim 1 wherein at least one of the leading balloon or the trailing balloon comprise a soft silicon rubber.

9. The robot of claim 1 wherein at least one of the leading balloon or the trailing balloon has a deflated external diameter in a range of 12 mm to 20 mm.

10. The robot of claim 1 wherein at least one of the leading balloon or the trailing balloon has an inflated external diameter of up to at least 90 mm.

11. The robot of claim 1 wherein at least one of the leading balloon or the trailing balloon has an activation pressure of 100 kPa or below.

12. The robot of claim 1 wherein the cavity has a diameter in a range of 1 mm to 8 mm.

13. The robot of claim 1 wherein the actuator has an external diameter in a range of 12 mm to 20 mm.

14. The robot of claim 1 wherein the chambers each have a length in a range of 12 mm to 30 mm.

15. The robot of claim 1 further comprising fluid control tubes fluidly connected to each of the leading balloon, trailing balloon and plurality of chambers.

16. The robot of claim 1 comprising a camera.
